(19) United States
(12) Patent Application Publication
(10) Pub. No.: US 2009/0265015 A1
(43) Pub. Date: Oct. 22, 2009

(54) Method And Apparatus For Attaching Soft Tissue To Bone

(75) Inventors: Brian M. May, Warsaw, IN (US); Mark A. Bollinger, Fort Wayne, IN (US)

Correspondence Address: HARNESS, DICKEY & PIERCE, P.L.C., P.O. BOX 828, BLOOMFIELD HILLS, MI, 48303, US

(73) Assignee: BIOMET MANUFACTURING CORP., Warsaw, IN (US)

(21) Appl. No.: 12/107,437

(22) Filed: Apr. 22, 2008

(51) Int. Cl. A61F 2/36

(52) U.S. Cl. 623/23.26

(57) ABSTRACT

A system and method for attaching soft tissue to a prosthetic implant can include a prosthetic component that defines a soft tissue attachment region having an attachment surface thereon. A first construct can be formed of porous metal and be removably coupled to the attachment surface. A second construct can be positioned outboard of the soft tissue. A fastener can be engaged to the second construct and capture the soft tissue against the first construct. The fastener can be coupled on a distal end to the first prosthetic component.

### FIELD

The present disclosure relates to endoscopic soft tissue fixation. More particularly, the present disclosure relates to an apparatus and a method for securing soft tissue to bone.

### INTRODUCTION

Ligaments and tendons are soft collagenous tissues. Ligaments are strong fibrous connective soft tissue, which connect the articular ends of bones to bind them together and to facilitate or limit motion. Tendons connect muscle to bone. Injuries to ligaments are common, and patients who are physically active are generally more susceptible to such ligament injuries. The anterior cruciate ligament (ACL) of the knee joint is a ligament frequently injured by such patients. Such injuries cause instability in the knee joint which, when left untreated, may lead to degenerative arthritis. Because of this condition, ACL reconstruction may be required. Generally during ACL reconstruction, a substitute soft tissue ligament or graft can be attached to the femur and/or tibia to facilitate regrowth and permanent attachment. The medial collateral ligament (MCL) and the lateral collateral ligament (LCL) are other ligaments associated with the knee that can become injured. Generally, injuries to the MCL occur more frequently than injuries to other ligaments of the knee. Isolated injuries of the LCL are less common and typically occur in association with ACL or posterior cruciate ligament (PCL) injuries.

When an injury (i.e., a tear) to any of these ligaments occurs, a surgical procedure can often be performed to repair the injury. During such a procedure it may be necessary to attach soft tissue (such as the host ligament, or a replacement graft) to bone. When using a replacement graft, the graft can be generally taken from the hamstring ligament, specifically, the semitendinosus and gracilis ligaments or tendons. When repairing an ACL or PCL, such grafts can be generally fed through a ligament tunnel and secured outside the tunnel. The graft can generally be secured by a non-endoscopic means of stapling or screwing the graft onto the outside surface of the tibia and/or femur.

In other examples, it may be necessary to reattach damaged or deficient ligaments to a total joint replacement component during implantation of a given replacement prosthesis. In instances where femoral replacement is necessary but some or all of the MCL, LCL and/or PCL are intact; it may be suitable to implant a cruciate retaining femoral component. In some examples, it may be desirable to reattach ligaments to preserve host bone, reduce trauma to the patient and prolong the need for more constraining devices.

### SUMMARY

An assembly for attaching soft tissue to a prosthetic implant can include a prosthetic component that defines a soft tissue attachment region having an attachment surface thereon. A first construct can be formed of porous metal and be removably coupled to the attachment surface. A second construct can be positioned outboard of the soft tissue. A fastener can be engaged to the second construct and capture the soft tissue against the first construct. The fastener can be coupled on a distal end to the first prosthetic component.

According to additional features, the prosthetic component can be a femoral component. The first construct can define a first washer having a first aperture formed therein. The first washer can define a plurality of extension portions formed on an outboard surface that extend in a direction away from the prosthetic component. The second construct can define a second washer having a second aperture formed therein. The second washer can define notches formed therein, wherein the plurality of extension portions extend through the notches in an assembled position. According to one example, the notches can be defined around a perimeter of the second washer. The fastener can threadably engage a threaded bore formed in the prosthetic component in an assembled position.

According to additional features, the extension portions can pierce the soft tissue in an assembled position. The extension portions can define spikes according to one example or raised walls according to another example. According to one example, the first and second washers can each define an arcuately shaped body that conforms to the attachment surface of the prosthetic component. The soft tissue attachment region can be defined on a lateral surface of a lateral condyle of the femoral component and the soft tissue can be a lateral collateral ligament (LCL). According to additional features, the soft tissue attachment region can be defined on a medial surface of a medial condyle of the femoral component and the soft tissue can be a medial collateral ligament (MCL). According to still another example, the soft tissue attachment region can be defined on an interior wall surface of the femoral component and the soft tissue can be either an anterior cruciate ligament (ACL) or posterior cruciate ligament (PCL).

A method for securing a soft tissue to a first prosthetic implant can include providing a first prosthetic component that defines a threaded bore at a soft tissue attachment region. A first construct formed of porous metal can be provided that defines a first aperture. A second construct can be provided that defines a second aperture. A soft tissue can be positioned between the first construct and the second construct. A fastener can be advanced through the second aperture and the first aperture. The fastener can be further advanced into the threaded bore, such that the soft tissue is progressively captured between the first and second constructs.

The method according to additional features of the present application can include removing the fastener from the first prosthetic component. The first construct can be removed from the first prosthetic component. The first construct can have the soft tissue ingrown within the porous material. The first prosthetic component can be replaced with a second prosthetic component having a second soft tissue attachment region. The first construct with the soft tissue ingrown within the porous material can be located at the second soft tissue attachment region. A fastener can be advanced through the second aperture and the first aperture. The fastener can be further advanced into a second threaded bore defined on the second prosthetic component.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

The skilled artisan will understand that the drawings, described below, are for illustration purposes only. The drawings are not intended to limit the scope of the present teachings in any way:

FIG. 1 is perspective anterior/lateral view of a right knee having a soft tissue securing assembly associated with a lateral collateral ligament (LCL) according to one example of the present teachings;

FIG. 2 is perspective anterior/lateral view of a right knee having a soft tissue securing assembly associated with a lateral collateral ligament (LCL) according to another example of the present teachings;

FIG. 3 is an exploded view of the soft tissue securing assembly of FIG. 1 and including an exemplary fastener, outboard washer and inboard washer;

FIG. 4 is a posterior perspective view of a femoral component shown with an exemplary securing assembly in exploded view;

FIG. 5 is a posterior perspective view of a femoral component shown with another exemplary securing assembly in exploded view;

FIG. 6 is an exploded perspective view of exemplary securing assemblies according to the present teachings;

FIG. 7 is an exploded side view of an exemplary securing assembly wherein the fastener is configured to threadably mate with a threaded bore formed in the prosthesis component according to a first example;

FIG. 8 is an exploded side view of an exemplary securing assembly wherein the fastener is configured to threadably mate with a threaded bore formed in the inboard washer according to a second example;

FIG. 9 is an exploded side view of an exemplary securing assembly wherein a porous metal portion is integrally formed in the prosthesis component according to one example; and

FIG. 10 is an exploded side view of an exemplary securing assembly wherein spikes are machined onto the prosthesis component according to one example.

### DETAILED DESCRIPTION OF VARIOUS EMBODIMENTS

The following description of the embodiment(s) is merely exemplary in nature and is in no way intended to limit the disclosure, its application, or uses. Moreover, while the present teachings are discussed in detail below with regard to attaching soft tissue to a knee component, those skilled in the art will recognize the other types of soft tissue attachment to other joints such as, but not limited to, a hip (proximal femoral component), elbow (proximal ulnar component, distal humerus component) or shoulder (proximal humerus component), or other areas of the body may employ the present teachings.

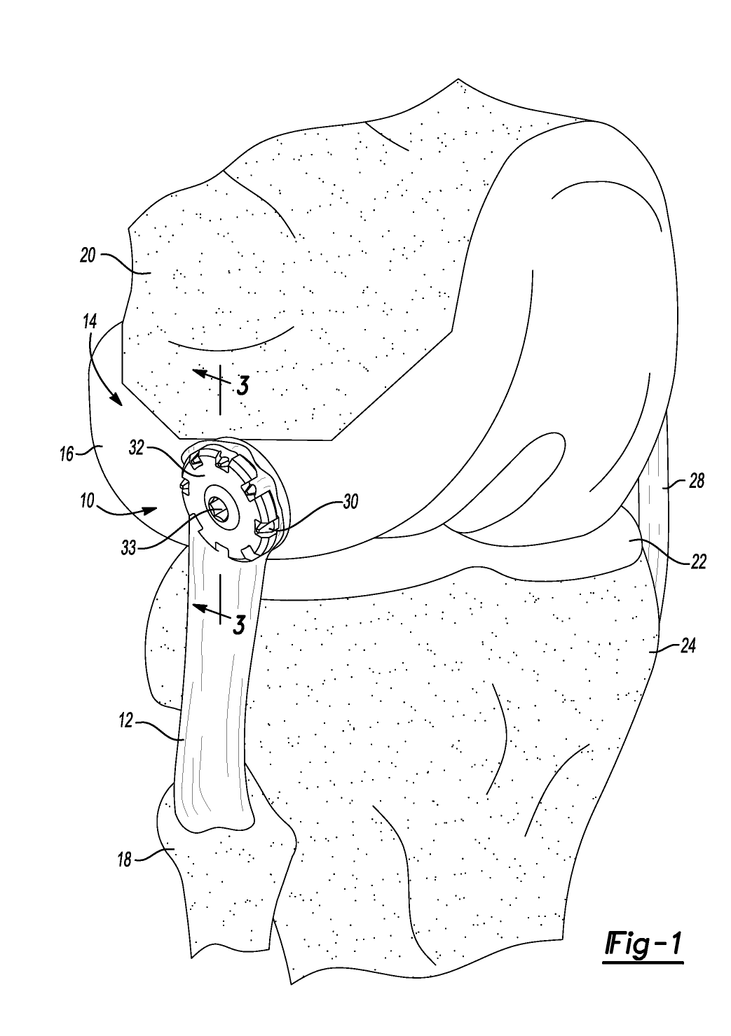

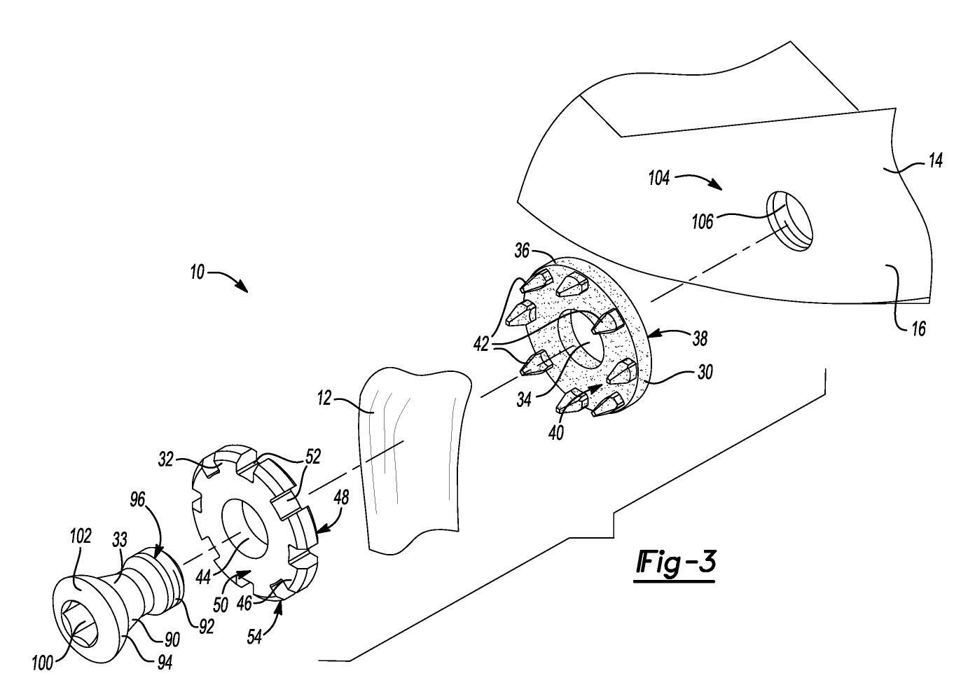

With initial reference to FIGS. 1 and 3, a securing assembly 10 according to the present teachings is illustrated and shown operatively securing a soft tissue 12 to a prosthetic implant 14. In the particular example shown, the soft tissue 12 is a lateral collateral ligament (LCL) and the prosthetic implant 14 is a femoral component 16. The LCL is naturally secured on one end to a fibula 18 and on the other end to the femoral component 16 by way of the securing assembly 10. The femoral component 16 is shown implanted on a distal femur 20. A tibial component 22 can also be implanted onto a tibia 24. While not particularly shown, a similar securing assembly can be provided at the medial side of the femoral component 16 for securing a medial collateral ligament (MCL) 28.

The securing assembly 10 can generally define a first construct or washer 30, a second construct or washer 32 and a fastener 33. The first washer 30 can define a first aperture 34. In one example, the first aperture 34 can be a centrally defined through-hole. The first washer 30 can define a disk shaped body 36 having an inboard face 38 and an outboard face 40. A plurality of extension portions 42 can be defined around the outboard face 40 of the first washer 30. According to the example shown in FIG. 3, the extension portions 42 are in the form of spikes. The second washer 32 can define a second aperture 44. In one example, the second aperture 44 can be a centrally defined through-hole, The second washer 32 can define a disk shaped body 46 having an inboard face 48 and an outboard face 50. A plurality of notches 52 can be formed around a perimeter 54 of the second washer 32. In one example, the plurality of notches 52 can be configured to accept the plurality of extension portions 42 in an assembled position (FIG. 1).

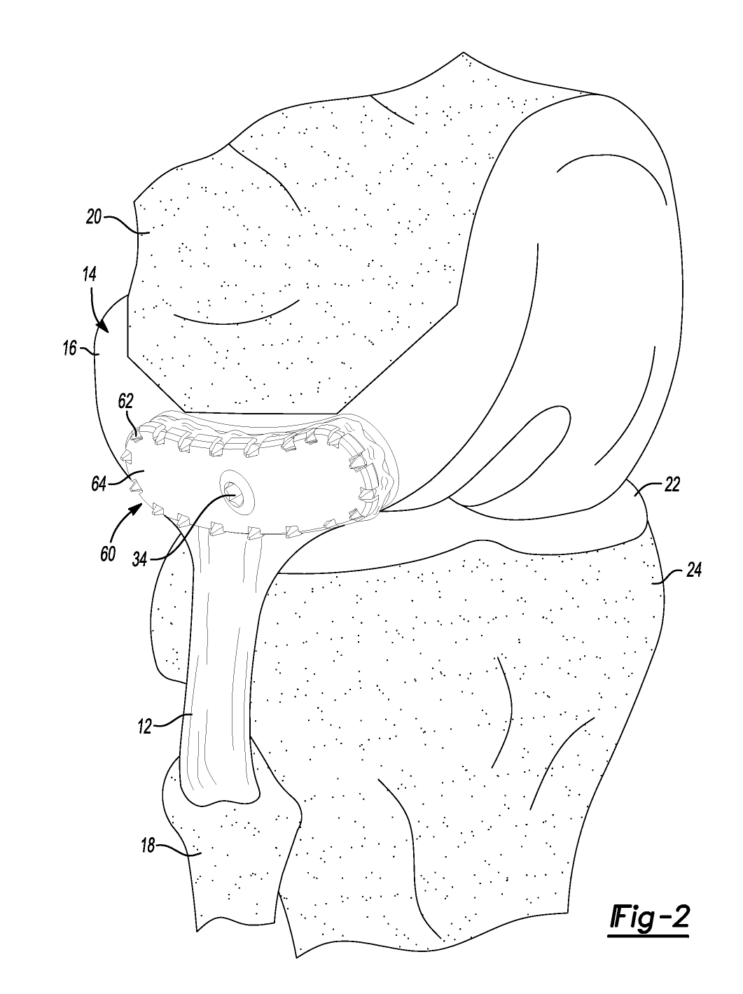

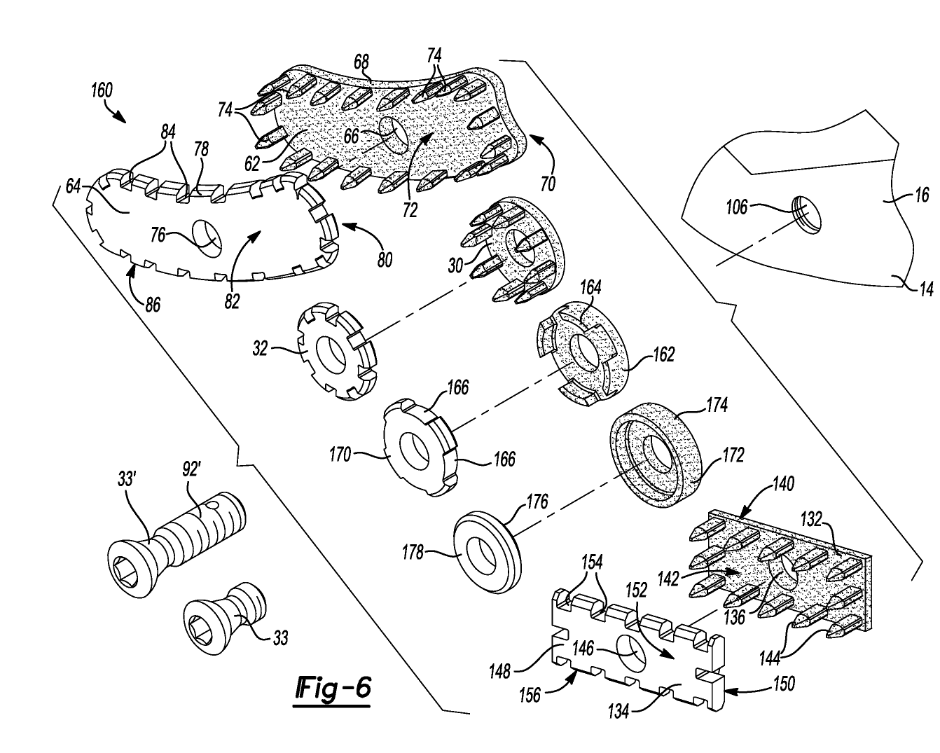

In the example shown in FIGS. 1 and 3, both of the first and second washers 30 and 32 define a body 36 and 46 that is substantially planar. As illustrated in FIG. 2, a securing assembly 60 according to additional features is shown having a generally arcuate profile as will be described in greater detail herein. The securing assembly 60 generally includes a first construct or plate 62, a second construct or plate 64 and a fastener 33. The first plate 62 can define a first aperture 66 (FIG. 6). In one example, the first aperture 66 can be a centrally defined through-hole. The first plate 62 can define a curved body 68 having an inboard face 70 and an outboard face 72. A plurality of extension portions 74 can be defined around the outboard face 72 of the first plate 62. According to the example shown in FIG. 2, the extension portions 74 are in the form of spikes. The second plate 64 can define a second aperture 76. In one example, the second aperture 76 can be a centrally defined through-hole. The second plate 64 can define an arcuate body 78 having an inboard face 80 and an outboard face 82. A plurality of notches 84 can be formed around a perimeter 86 of the second plate 64. In one example, the plurality of notches 84 can be configured to accept the plurality of extension portions 74 in an assembled position (FIG. 2).

In one example, the first construct 30, 62 can be formed of porous biocompatible material. The porous biocompatible material can include porous titanium. In general, the porous material used herein can provide pores that are irregular in size and orientation. Other suitable porous biocompatible materials can be found in co-owned and co-pending U.S. patent application Ser. No. 11/357,929, which is expressly incorporated by reference herein. The porous biocompatible material encourages tissue ingrowth of the soft tissue 12.

In one example, the second construct 32, 64 can be formed of solid biocompatible material such as titanium for example. According to additional features, the second construct 32, 64 can be alternately formed of porous biocompatible material. The porous biocompatible material can include porous titanium or other porous biocompatible materials referenced above.

Returning now to FIG. 3, the fastener 33 can define an intermediate portion 90 extending between a distal end 92 and a proximal end 94. The distal end 92 can define threads 96. The proximal end 94 can define a tool engagement feature 100. The tool engagement feature 100 can be any suitable tool engagement detail such as a hex-head for example. The proximal end 94 can further define a conical body portion 102. The conical body portion 102 can suitably nest within the second aperture 44 (or 76) of the second washer 32 (or plate 64). In this way, the proximal end 94 of the fastener 33 can rest substantially flush with the outboard surface 50 of the second washer 32 (or outboard face 82 of plate 64).

With continued reference to FIG. 3, the prosthetic implant 14 can define an attachment region 104 having a threaded bore 106 therein. The threaded bore 106 can be configured to threadably mate with the threads 96 at the distal end 92 of the fastener 33. In other examples, the attachment region 104 can be defined on the host bone and the fastener 33 can be a bone screw for securing the assembly onto the host bone.

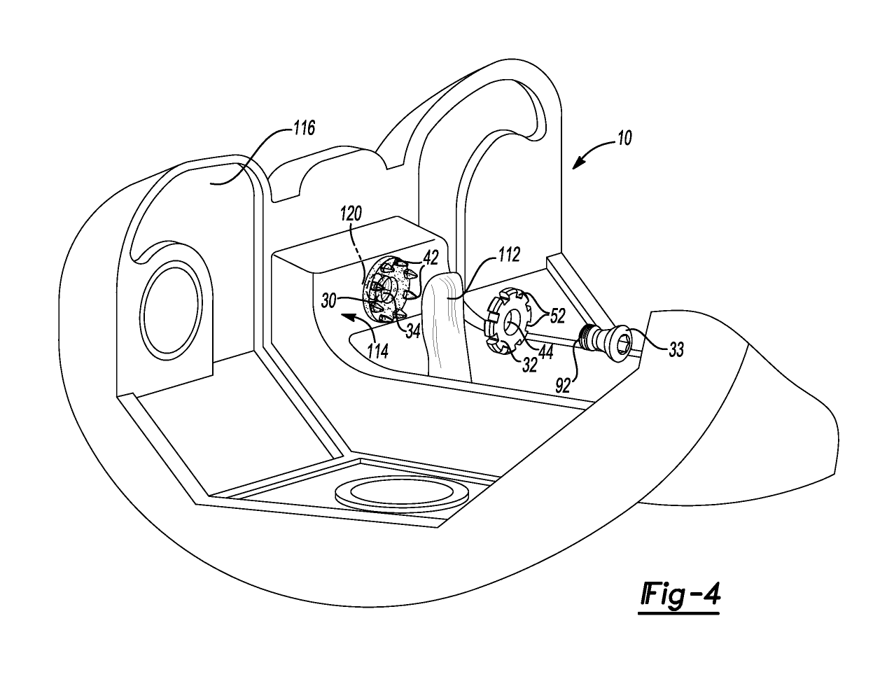

With reference now to FIG. 4, the securing assembly 10 is shown securing a posterior cruciate ligament (PCL) or an anterior cruciate ligament (ACL) 112 to an inboard wall 114 of a femoral component 116. The inboard wall 114 of the femoral component 116 can define a threaded bore 120 that threadably receives the distal end 92 of the fastener 33.

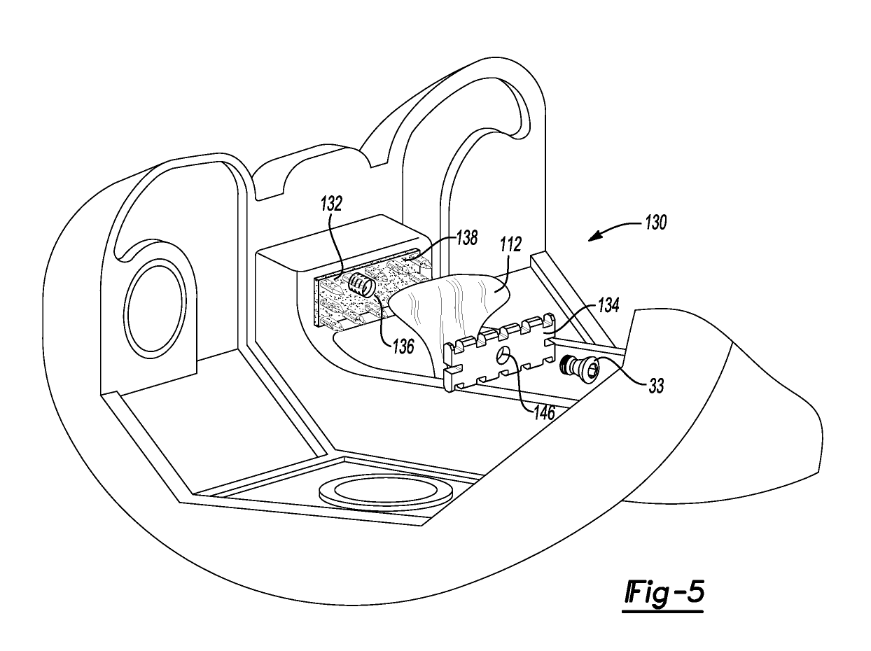

As illustrated in FIG. 5, a securing assembly 130 according to additional features is shown having a generally planar, rectangular profile as will be described in greater detail below. The securing assembly 130 can generally include a first construct or plate 132, a second construct or plate 134 and a fastener 33. The first plate 132 can define a first aperture 136. In one example, the first aperture 136 can be a centrally defined through-hole. The first plate 132 can define a planar body 138 having an inboard face 140 (FIG. 6) and an outboard face 142. A plurality of extension portions 144 can be defined around the outboard face 142 of the first plate 132. According to the example shown in FIG. 5, the extension portions 144 are in the form of spikes. The second plate 134 can define a second aperture 146. In one example, the second aperture 146 can be a centrally defined through-hole. The second plate 134 can define a planar body 148 having an inboard face 150 and an outboard face 152. A plurality of notches 154 can be formed around a perimeter 156 of the second plate 134. In one example, the plurality of notches 154 can be configured to accept the plurality of extension portions 144 in an assembled position (similar to that shown with the securing assembly 10 in FIG. 1).

With reference now to FIG. 6, a plurality of attachment assemblies 160 are shown. In addition to the first and second constructs described above, a first construct 162 can be provided having a plurality of raised walls 164. The raised walls 164 can interfit within notches 166 defined around a second construct 170. A first construct 172 can define a continuous wall 174 that can surround a perimeter of a second construct 178. Another screw 32′ can define a longer distal end 92′. As can be appreciated, a kit of attachment assemblies can be provided such that a surgeon can easily select a suitable prosthetic implant, first construct, second construct and fastener for a particular application.

An exemplary method of attaching soft tissue to a prosthetic implant 14 using one of the attachment assemblies 160 will now be described. At the outset, a surgeon can select a suitable first construct (30 etc.), second construct (32 etc.) and fastener (33 etc.) for a particular application. Concurrently, a surgeon can select a desired prosthetic implant 176, such as the femoral components 16 provided herein. The first construct (30, etc.) can then be positioned adjacent to the attachment region 104 of the prosthetic implant 14. The soft tissue (12 etc.) can then be positioned intermediate of the outboard face (40 etc.) of the first construct (30 etc.) and the inboard face (48 etc) of the second construct (32 etc.). At this point, the extension portions (42 etc.) can be aligned with the notches (52 etc.). The fastener (33 etc.) can then be passed through the second aperture (44 etc.) of the second construct (32 etc.), pierced through the soft tissue (12 etc.), passed through the first aperture (34 etc.) of the first construct (30 etc.) and threaded into the threaded bore 106. As the fastener 33 is progressively threaded into the threaded bore 106, the extension portions (42 etc.) can pierce through the soft tissue (12 etc.) further capturing the soft tissue (12 etc.) between the first and second constructs (30 and 32 etc.).

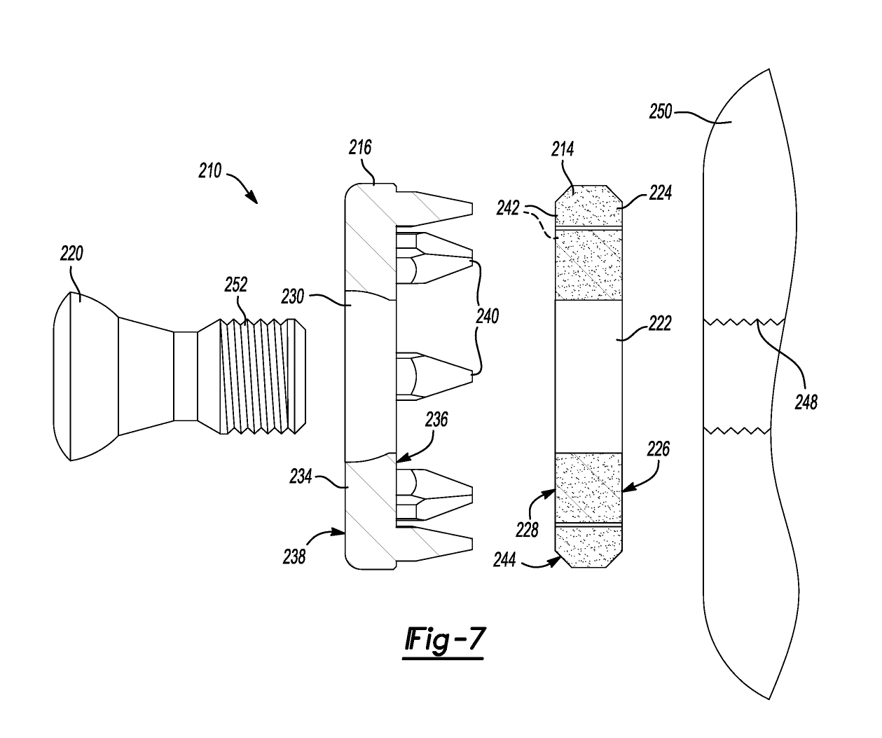

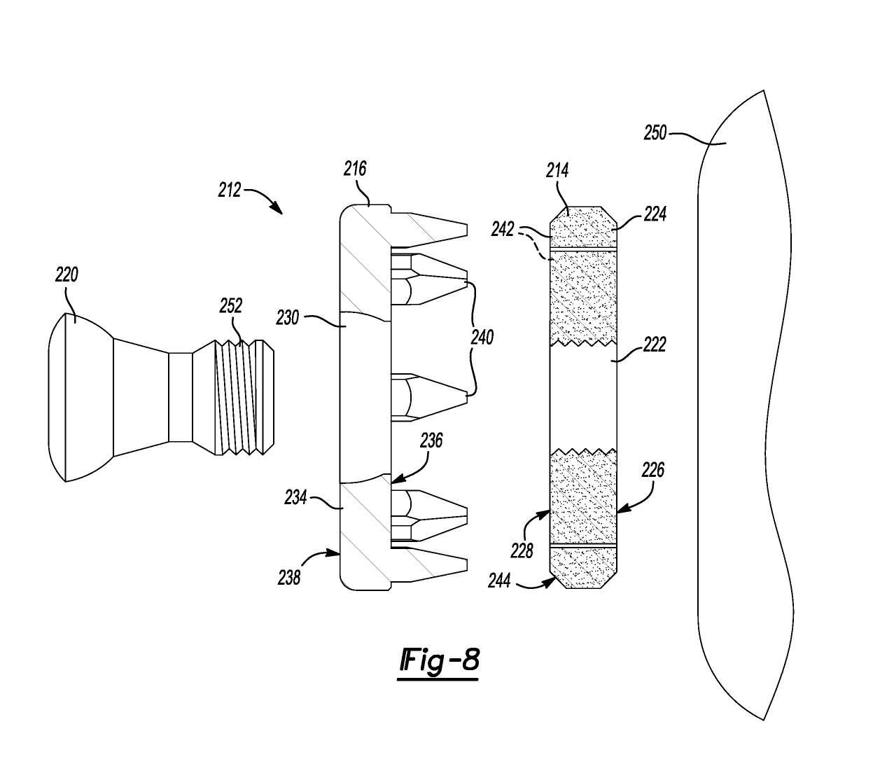

FIGS. 7 and 8 show securing assemblies 210 and 212 according to additional features. The securing assembly 210 generally includes a first construct or plate 214, a second construct or plate 216 and a fastener 220. The first plate 214 can define a first aperture 222. In one example, the first aperture 222 can be a centrally defined through-hole. The first plate 214 can define a planar body 224 formed of porous metal such as described herein. The first plate 214 can have an inboard face 226 and an outboard face 228. The second plate 216 can define a second aperture 230. In one example, the second aperture 230 can be a centrally defined through-hole. The second plate 216 can be formed of a solid metal or a porous metal as described herein. The second plate 216 can define a planar body 234 having an inboard face 236 and an outboard face 238. A plurality of extension portions 240 can be defined around the inboard face 236 of the second plate 216. According to the example shown in FIG. 7, the extension portions are in the form of spikes. A plurality of notches 242 can be formed around a perimeter 244 of the first plate 214. In one example, the plurality of notches 242 can be configured to accept the plurality of extension portions 240 in an assembled position. A threaded bore 248 can be defined in a prosthesis 250 for threadably receiving the distal end 252 of the fastener 220.

The securing assembly 212 shown in FIG. 8 is constructed substantially similar to the securing assembly shown in FIG. 7 except a threaded aperture 260 of a first construct 262 is threaded. The threaded aperture 260 is configured to threadably accept a distal end 264 of the fastener 266 in an assembled position. The inboard face of the first construct 262 can be secured to the prosthesis 250 by an adhesive such as bone cement. Other methods may be used to secure the first construct 262 to the prosthesis 250.

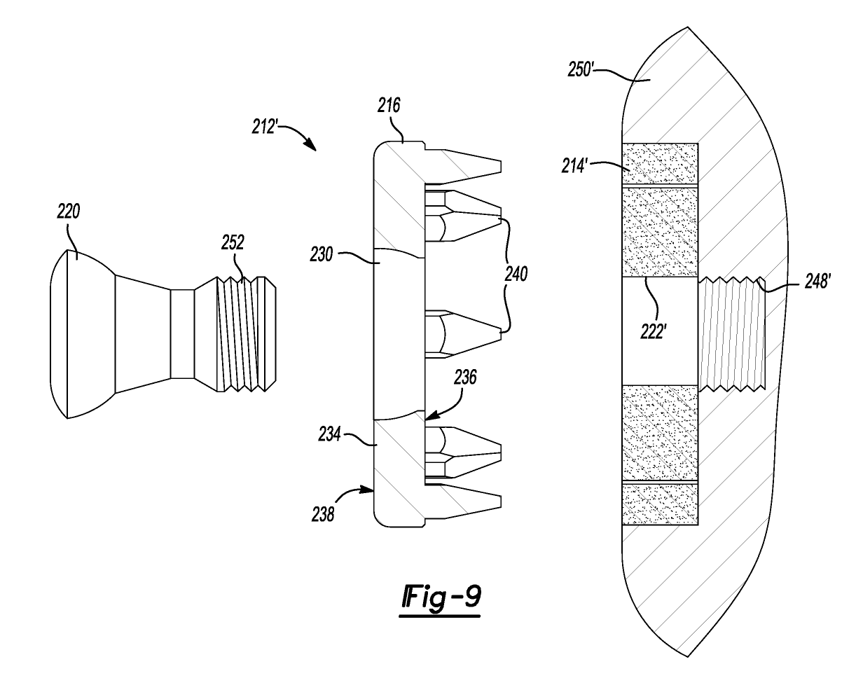

FIG. 9 shows a securing assembly 212′ according to additional features. The securing assembly 212′ generally includes a first construct or plate 214′, a second construct or plate 216 and a fastener 220. The first plate 214′ can define an aperture 222′. In one example, the aperture 222′ can be a centrally defined through-hole. The first plate 214′ according to the example shown in FIG. 9 can be formed of porous metal, such as described herein and also be integrally formed with a prosthesis 250′. The prosthesis 250′ can define a threaded bore 248′ for threadably receiving the distal end 252 of the fastener 220.

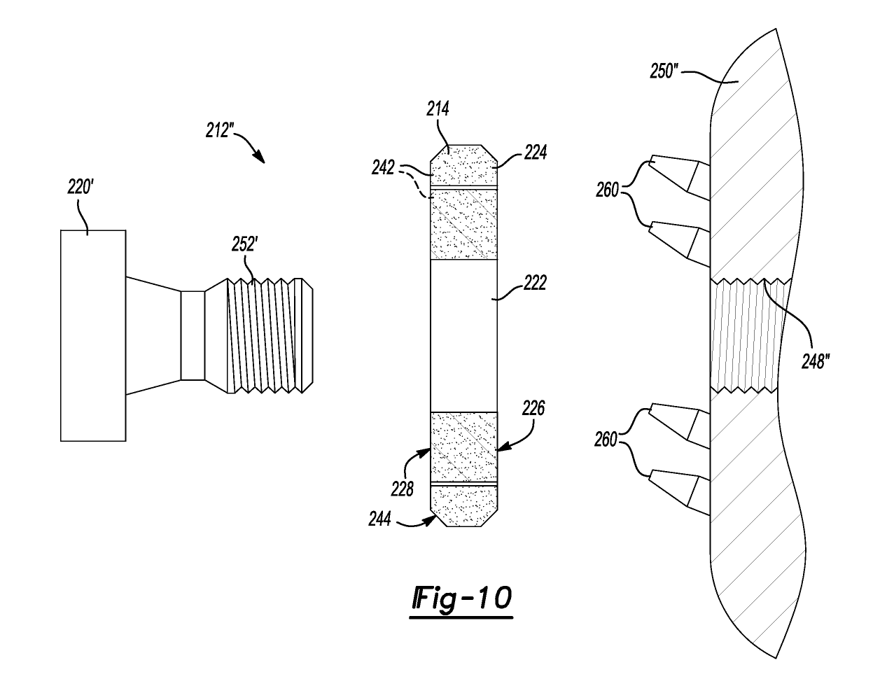

FIG. 10 illustrates a securing assembly 212″ according to additional features. The securing assembly 212″ generally includes a prosthesis 250″ having a threaded bore 248″ for threadably receiving a distal end 252′ of a fastener 220′. The prosthesis 250″ can include spikes 260 formed thereon. The spikes 260 can be integrally formed, such as by machining, with the prosthesis 250″. In one example, the spikes 260 can extend at an angle generally greater than 90° (obtuse) and in a direction away from an originating point of the cruciate ligament 112. The spikes 260 can therefore pierce the cruciate ligament 112 at a favorable angle for capturing the ligament 112 at the prosthesis 250″. In the example shown in FIG. 10, the need for a separate spiked washer can be eliminated.

According to one benefit of the instant disclosure, the soft tissue can have long term viability at the attachment region 104. The porous material of the first and/or the second construct (30 and 32 etc.) can facilitate substantial tissue ingrowth. In the event that the implant (i.e., the prosthetic implant 14) would need to be revised, the fastener (33 etc.) can be withdrawn from the threaded bore 106. The soft tissue (12 etc.) that has grown into the first construct (30 etc.) and second construct (32 etc.), can be collectively removed and or excised. A new prosthetic implant can then be implanted and the first and second construct (30 and 32 etc.) with ingrown soft tissue (12 etc.) can be re-fastened to the replacement implant.

Those skilled in the art can now appreciate from the foregoing description that the broad teachings of the present disclosure can be implemented in a variety of forms. Therefore, while this disclosure has been described in connection with particular examples thereof, the true scope of the disclosure should not be so limited since other modifications will become apparent to the skilled practitioner upon a study of the drawings, the specification and the following claims.

What is claimed is:

1. An assembly for attaching soft tissue to a prosthetic implant comprising:
a prosthetic component defining a soft tissue attachment region having an attachment surface thereon;
a first construct removably coupled to the attachment surface;
a second construct positioned outboard of the soft tissue; and
a fastener engaged to the second construct and capturing the soft tissue against the first construct, the fastener coupled on a distal end to the first prosthetic component, wherein at least one of the first and second constructs are formed of porous metal.

2. The assembly of claim 1 wherein the prosthetic component is a femoral component.

3. The assembly of claim 1 wherein the first construct defines a first washer having a first aperture formed therein.

4. The assembly of claim 3 wherein the first washer defines a plurality of extension portions formed on an outboard surface that extend in a direction away from the prosthetic component.

5. The assembly of claim 4 wherein the second construct defines a second washer having a second aperture formed therein.

6. The assembly of claim 5 wherein the second washer defines notches formed therein, wherein the plurality of extension portions extend through the notches in an assembled position.

7. The assembly of claim 6 wherein the notches are defined around a perimeter of the second washer.

8. The assembly of claim 6 wherein the fastener threadably engages a threaded bore formed in the prosthetic component in the assembled position.

9. The assembly of claim 6 wherein the extension portions pierce the soft tissue in the assembled position.

10. The assembly of claim 9 wherein the extension portions define at least one of spikes or raised walls.

11. The assembly of claim 6 wherein the first and second washers define an arcuately shaped body that conforms to the attachment surface of the prosthetic component.

12. The assembly of claim 3 wherein the first construct is formed of porous metal and the second construct defines a plurality of extension portions formed on an inboard surface that extends in a direction toward the prosthetic component.

13. The assembly of claim 2 wherein the soft tissue attachment region is defined on a lateral surface of a lateral condyle of the femoral component and wherein the soft tissue is a lateral collateral ligament (LCL).

14. The assembly of claim 2 wherein the soft tissue attachment region is defined on a medial surface of a medial condyle of the femoral component and wherein the soft tissue is a medial collateral ligament (MCL).

15. The assembly of claim 2 wherein the soft tissue attachment region is defined on an interior wall surface of the femoral component and wherein the soft tissue is one of an anterior cruciate ligament (ACL) or a posterior cruciate ligament (PCL).

16. The assembly of claim 1 wherein the first and second constructs define shapes selected from the group comprising circular, rectangular, and oblong.

17. A method for securing a soft tissue to a first prosthetic implant, the method comprising:
providing a first prosthetic component defining a threaded bore at a soft tissue attachment region;
providing a first construct formed of porous metal and defining a first aperture;
providing a second construct defining a second aperture;
positioning a soft tissue between the first construct and the second construct;
advancing a fastener through the second aperture and the first aperture; and
further advancing the fastener into the threaded bore such that the soft tissue is progressively captured between the first and second constructs.

18. The method of claim 17 wherein the further advancing the fastener includes advancing a plurality of extension portions formed on one of the first or second constructs into a plurality of notches formed on the other of the first and second constructs.

19. The method of claim 18 wherein the further advancing the fastener includes piercing the soft tissue with the extension portions.

20. The method of claim 18 wherein providing the first construct includes providing a first construct integrally formed with the first prosthetic component.

21. The method of claim 17 wherein positioning the soft tissue include piercing the soft tissue with at least one piercing member extending at an obtuse angle in a direction generally away from an originating point of the soft tissue.

22. The method of claim 18, further comprising:
removing the fastener from the first prosthetic component;
removing the first construct from the first prosthetic component, the first construct having the soft tissue ingrown within the porous metal;
replacing the first prosthetic component with a second prosthetic component having a second soft tissue attachment region;
locating the first construct with the soft tissue ingrown within the porous metal at the second soft tissue attachment region;
advancing a fastener through the second aperture and the first aperture; and
further advancing the fastener into a second threaded bore defined on the second prosthetic component.

23. The method of claim 17 wherein providing the prosthetic component includes providing a femoral component wherein the tissue attachment region is defined on a lateral surface of a lateral condyle of the femoral component and wherein the soft tissue is a lateral collateral ligament (LCL).

24. The method of claim 17 wherein providing the prosthetic component includes providing a femoral component wherein the tissue attachment region is defined on a medial surface of a medial condyle of the femoral component and wherein the soft tissue is a medial collateral ligament (MCL).

25. The method of claim 17 wherein providing the prosthetic component includes providing a femoral component wherein the tissue attachment region is defined on an interior wall surface of the femoral component and wherein the soft tissue is one of an anterior cruciate ligament (ACL) or a posterior cruciate ligament (PCL).
